(19) United States
(12) Patent Application Publication
(10) Pub. No.: US 2024/0287289 A1
(43) Pub. Date: Aug. 29, 2024

(54) STERILIZED MULTICOMPONENT COMPOSITION FOR REMOVAL OF PARTICLES

(71) Applicant: PURENUM GMBH, Bremen (DE)

(72) Inventors: Ingo GRUNWALD, Lilienthal (DE); Sebastian STÖßLEIN, Bremen (DE)

(73) Assignee: PURENUM GMBH, Bremen (DE)

(21) Appl. No.: 18/565,881

(22) PCT Filed: Jun. 9, 2022
(86) PCT No.: PCT/EP2022/065643

§ 371 Date: Nov. 30, 2023

(30) Foreign Application Priority Data
Jun. 9, 2021 (DE) 20 2021 103 106.9

(51) Int. Cl. C08L 5/04; A61K 9/06; A61K 47/36; C08J 3/075; C08J 3/24; C08K 3/16; C08K 3/32; C08K 5/3465

(52) U.S. Cl. CPC C08L 5/04; A61K 9/06; A61K 47/36; C08J 3/075; C08J 3/24; C08K 3/16; C08K 3/32; C08K 5/3465; C08J 2305/04; C08K 2003/162; C08K 2003/324

(57) ABSTRACT

The present invention relates to a sterilized, gel-forming, multi-component composition comprising a component containing at least one crosslinkable polymer and a component containing at least one crosslinking agent. Further, the present invention relates to such a composition or gel for use in a method of removing undesirable particles from a patient, as well as to a method of making such a composition and a composition producible or produced by such a method.

The present invention relates to a sterilized, gel-forming, multi-component composition comprising a component containing at least one crosslinkable polymer and a component containing at least one crosslinking agent. Further, the present invention relates to such a composition or gel for use in a method of removing undesirable particles from a patient, as well as to a method of making such a composition and a composition producible or produced by such a method.

Further aspects of the present invention or in connection therewith, and preferred embodiments are described below and in the appended claims.

The accumulation of undesirable precipitates or deposits of various kinds are a common problem in the human body. Known forms of these include gallstones or kidney stones. Gallstones occur in about 10 to 15% of the adult population, with western industrialized countries being particularly affected. About half of the German population over the age of 60 has gallstones. While gallstones often remain asymptomatic, some cases develop colic with severe pain, elevated liver enzymes, and general symptoms of disease, including jaundice (icterus).

From an epidemiological point of view, kidney stone disease is one of the most common diseases of mankind, with an incidence of 1.45% in Germany in 2000, which in turn corresponds to approximately 1,200,000 new cases per year. In Germany alone, a total of approximately 750,000 treatment cases per year can be assumed. The number of treatments for stone removal in Germany is estimated at about 400,000/year, of which about half are for treatments of recurrent stones. These FIGURES can be extrapolated to the millions of such treatments performed worldwide. With a sum of over 1.5 billion euros, kidney stone disease thus represents a considerable cost factor in the German health care system. Kidney stones can also cause severe pain, trigger kidney inflammation, damage the kidneys or even lead to (usually unilateral) acute kidney failure.

The causes of both problems are varied and range from an unhealthy, high-fat diet or dehydration or lack of exercise, to diseases such as diabetes mellitus or gout, to genetic predisposition.

Frequently, gallstones or kidney stones are only discovered at an already advanced stage, usually when the first (more serious) symptoms appear. In most cases, the stones that have formed are then already too large to be removed from the body in one piece by minimally invasive procedures. In this case, the stones that have formed are crushed and the individual fragments of them are removed. In some cases, several stones are formed, which also need to be completely removed.

A method of treating kidney stones by selective dissolution of the deposits using quaternary ammonium salts is described, for example, in U.S. Pat. No. 5,244,913.

It is not only in the case of gallstones or kidney stones or other stones in the body that several unwanted particles must be removed from a patient. In other cases, too, for example in the case of comminuted fractures, in this case splinters of the body's own bone, or abrasions, in this case foreign bodies such as stones, metal, plastic or wood splinters or fragments thereof, a large number of particles must be removed from the patient as completely as possible.

All these cases have in common that a large number of particles must be removed from the patient as uncomplicatedly as possible, but at the same time as completely as possible.

If, for example, the gallstones or kidney stones do not leave the body naturally or if there are medical indications for immediate therapy, endoscopy (minimally invasive mirror techniques) represents the therapeutic “gold standard” along with extracorporeal shock wave treatment (ESWL). In view of the increasing evidence for worse results of ESWL, endoscopic procedures are preferred. It is estimated that currently 60-70% of all stone patients are treated endoscopically. This tendency is increasing. With the aid of endoscopic techniques, kidney stones are crushed and removed on site. An as yet unsolved problem is posed in particular by small residual fragments (<2 mm), which cannot be effectively removed during treatment. Remaining kidney stone fragments act as “crystallization nuclei” from which new stones develop in up to 70% of cases. This in turn leads to renewed medical problems and need for treatment. Such fragments were previously referred to as clinically irrelevant residual fragments (CIRF), although it has become clear in recent years that they are very much clinically relevant.

In lithotripsy, kidney stones are fragmented by extracorporeal shock waves or endoscopically introduced laser or compressed air probes. This produces fragments of varying sizes that can either be removed using barrel instruments or flushed out. A problem encountered in lithotripsy is that fragments can disperse during fragmentation or reach regions that are difficult to access.

WO 2005/037062 relates to a method of entrapping (not enclosing) kidney stones in a specific area using a polymer plug, which can largely prevent tissue damage from the resulting fragments during fragmentation. According to WO 2005/037062, a gel-forming liquid is injected into the lumen on at least one side of a kidney stone, for example a thermosensitive polymer that forms a gel plug at body temperature. The polymer does not usually come into contact with the kidney stone, but serves to increase the efficiency of the lithotripsy by preventing the kidney stone from shifting, and protects the surrounding tissue from damage due to fragmentation. Application of this system is explicitly recommended or allowed only outside the kidney.

An approach to remove objects, such as blood clots, from the body using an adhesive is given in US 2008/0065012. In this approach, the adhesive is spread on a surface and introduced into the body using a catheter. When the object is adhered to the surface, the catheter is withdrawn, taking the object with it.

Adhesives based on biological macromolecules and, in particular, gel-forming polymer systems are increasingly finding applications in medical technology. Their high biocompatibility is one of the most important selection criteria.

In U.S. Pat. No. 6,663,594 B2 a method for immobilizing an object, for example a kidney stone, in the body, is described, in which a gel-forming liquid is injected into the body. Upon contact with the object, a gel is formed that at least partially engages and immobilizes the object. The immobilization serves to allow the object to be subsequently fragmented without risking distribution of the fragments or to allow the object or fragments to be removed from the body with an endoscopic tool. In doing so, the gel prevents the object or a fragment from slipping and from not being able to be captured by the tool. After removing the object or fragments, the gel is dissolved or extracted using an endoscopic tool. The disadvantage of this method is that when the kidney stones are fragmented, the already set gel may be destroyed, thereby releasing fragments again, or individual fragments may escape from the polymer. Gripping the fragments in the gel is not possible with conventional gripping tools. In addition, the procedure described is very laborious, since the stones or stone fragments are gripped and removed individually. As a result, individual stone fragments are relatively likely to remain.

A particular problem with lithotripsy is the occurrence of medium-sized stone fragments (especially <2 mm), also referred to as “grit”, as these fragments can neither be efficiently grasped nor rinsed. Residual fragments of this size slip through the meshes of grasping instruments (barrel forceps or baskets), making the extraction of grit very time-consuming and practically impracticable for larger stone masses. However, the retention of such kidney stone fragments leads to the formation of new kidney stones in a very high percentage of cases, as the fragments or fragments act as “crystallization nuclei”.

WO 2014/173467 describes a gel-forming system comprising a component containing one or more crosslinkable polymers, a component containing one or more crosslinking agents, and optionally a component containing magnetizable particles. The gel-forming system can be used to remove urinary stones. In this process, the urinary stone(s) is/are fragmented. Subsequently, the component containing one or more cross-linking agents and the component containing magnetizable particles are mixed and injected by catheter via an endoscope device into the area of the urinary tract containing the fragments of the crushed urinary stone(s). The component containing one or more crosslinkable polymers is then added, resulting in the formation of a gel. The solidified gel thereby partially or completely encloses the urinary stones or fragments thereof and can be removed together with the urinary stones or fragments thereof by means of a gripping instrument via the surgical endoscope.

The injection of components that form a gel after mixing often presents the difficulty of finding a suitable balance between a sufficiently high viscosity so that a gel can form particularly easily and a sufficiently low viscosity so that the respective component can be injected particularly well. The resulting gel should also have a suitable strength so that it can be removed again particularly easily. In addition, the time between mixing the components should be sufficiently low so that, for example, a medical procedure can be performed as quickly as possible. At the same time, however, the time should not be too short, otherwise there may not be sufficient mixing of the components and a very inhomogeneous gel may result, or lumps may form during gel formation.

Another difficulty is that the components often have to be sterilized for medical use. After sterilization, the components or constituents of the components often have only a greatly reduced function, especially for gel formation. The same applies to the storage of the components. Thus, in particular, the cross-linkable polymers of such components are degraded during sterilization, whereby their ability to subsequently form a gel is greatly reduced or even completely lost.

The primary object of the present invention, therefore, was to provide a possibility in which particles can be removed from a patient and in which the above advantages, or as many of these advantages as possible, can be achieved without the listed disadvantages, or with as few of the disadvantages as possible.

In particular, it was the task of the present invention to provide an improved, sterilized means that is suitable for reliably extracting particles from the body.

The primary problem of the present invention is solved by a sterilized gel-forming multi-component composition, preferably two-component composition, for forming a gel,

the composition consisting of or comprising
component (A), comprising
(i) one or more crosslinkable, preferably cationically crosslinkable, polymer(s), preferably wherein the or a polymer is sodium alginate,
(ii) water,
(iii) NaCl in a range of from 0.3 to 1.2 wt.-% based on the total weight of component (A)
(iv) one or more phosphate buffers and
(v) optionally: one or more dyes
and
component (B) comprising
(a) one or more crosslinking agent(s) for crosslinking the crosslinkable polymer(s) in component (i) of component (A), preferably wherein the or a cross-linking agent is CaCl₂), and
(b) water, and
(c) optionally: one or more dyes.

Preferably, component (i) of component (A) comprises sodium alginate and component (a) of component (B) comprises CaCl₂).

Preferably, component (i) of component (A) is sodium alginate and component (a) of component (B) is CaCl₂).

Sodium alginate is particularly suitable for use in the body as a crosslinkable polymer because it does not cause inflammatory reactions or immune rejections and carries a low risk of tissue trauma. It is also biodegradable. Crosslinking occurs rapidly but without sticking to fine tissue structures of the patient or the endoscopy instruments. The resulting gels have sufficient stability and flexibility to be extracted together with the particles.

Preferably, at least 40%, preferably at least 50%, preferably at least 60%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99% of the sodium alginate in component (A) have a molar mass of at least 110,000 g/mol, preferably at least 125,000 g/mol, preferably at least 150,000 g/mol, preferably at least 175,000 g/mol, preferably at least 200,000 g/mol, preferably at least 225,000 g/mol, preferably at least 250,000 g/mol, preferably at least 275,000 g/mol, preferably at least 300,000 g/mol, preferably at least 325,000 g/mol, preferably at least 350,000 g/mol, preferably at least 375,000 g/mol, preferably at least 400,000 g/mol.

Preferably, at least 40%, preferably at least 50%, preferably at least 60%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99% of the sodium alginate in component (A) have a molar mass of at most 700,000 g/mol, preferably at most 600,000 g/mol, preferably at most 550,000 g/mol, preferably at most 500. 000 g/mol, preferably maximum 475,000 g/mol, preferably maximum 450,000 g/mol, preferably maximum 425,000 g/mol, preferably maximum 400,000 g/mol, preferably maximum 380,000 g/mol, preferably maximum 360,000 g/mol, preferably maximum 350,000 g/mol, preferably maximum 340. 000 g/mol, preferably maximum 320,000 g/mol, preferably maximum 300,000 g/mol, preferably maximum 280,000 g/mol, preferably maximum 260,000 g/mol, preferably maximum 250,000 g/mol, preferably maximum 240,000 g/mol, preferably maximum 220,000 g/mol, preferably maximum 200,000 g/mol.

Any combination of the described minimum and maximum molar masses are hereby disclosed for any described amount in the alginate. The same is also true in the case where the minimum and maximum molar masses apply to a different amount of the alginate.

Alginate, or sodium alginate, is a polysaccharide built up from the two uronic acids guluronic acid (G) and mannuronic acid (M). In alginates, the proportion of G and the proportion of M in the alginate molecule can vary greatly. The proportion of G determines the proportion of M, i.e. if the proportion of G is 75%, the proportion of M is 25%.

Preferably, at least 40%, preferably at least 50%, preferably at least 60%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99% of the sodium alginate in component (A) have a G content of at least 30%, preferably at least 35%, preferably at least 40%, preferably at least 45%, preferably at least 50%, preferably at least 55%, preferably at least 60%, preferably at least 65%, preferably at least 70%, preferably at least 75%, preferably at least 80%, preferably at least 85%, preferably at least 90%, preferably at least 95%.

Preferably, at least 40%, preferably at least 50%, preferably at least 60%, preferably at least 70%, preferably at least 75%, preferably at least 80%, preferably at least 85%, preferably at least 90%, preferably at least 95%, preferably at least 96%, preferably at least 97%, preferably at least 98%, preferably at least 99% of the sodium alginate in component (A) have a G content of at most 99%, preferably at most 98%, preferably at most 97%, preferably at most 96%, preferably at most 95%, preferably at most 90%, preferably at most 85%, preferably at most 80%, preferably at most 75%, preferably at most 70%, preferably at most 65.

Any combination of the described minimum and maximum G proportions are hereby disclosed for any described amount in the alginate. The same is also true in the case where the minimum and maximum G fractions apply to a different amount of the alginate.

Also disclosed hereby are any combinations of the described minimum and/or maximum molar masses and the described minimum and/or maximum G proportions in the alginate. The same also applies in the case where the minimum and/or maximum molar mass and/or the minimum and/or maximum G fraction applies to a different amount of the alginate.

CaCl₂ provides naturally occurring cations in physiological systems that can be easily administered in the form of biologically compatible solutions. They have suitable coordination chemistry and can form stable chelate complexes for crosslinking and thus gel formation.

The term “gel-forming composition” as used herein is preferably understood to mean that the composition is not only capable of forming a gel, but that a gel is actually formed by mixing the components of the composition.

The gel formed by mixing the components of the composition is preferably a hydrogel.

It was surprisingly found that component (A), as described in the compositions according to the invention, can advantageously be sterilized, so that component (A) remains functional after the sterilization process has taken place. Thus, individual or the individual components or the multi-component composition can be sterilized to find application in the medical, e.g. invasive or surgical, field, in particular to be introduced into a patient.

The combination of NaCl and phosphate(s) in component (A), as described in the compositions according to the invention, significantly reduced the reduction of gel formation after sterilization (see Example 3). Surprisingly, it was found that in addition to the presence of phosphates in component (A), the concentration of NaCl is also important in allowing gel formation to occur advantageously even after sterilization has occurred. Surprisingly, phosphates as well as NaCl, as described in the compositions according to the invention, exert a synergistic effect on the preservation of the function after sterilization (cf. Example 3).

Preferably, the amount of NaCl is in a range of from 0.3 to 1.2 wt.-%, more preferably in a range of from 0.4 to 1.0 wt.-%, based on the total weight of component (A).

If one or more of the components of component (A), in addition to component (iii), should contain NaCl, the total NaCl present in component (A) is used to calculate the wt.-% of NaCl. For example, should the phosphate buffer in component (iv) of component (A) also contain NaCl, the NaCl of the phosphate buffer will also be used to calculate the wt.-% of NaCl in component (iii).

The term “phosphate buffer” as used herein describes a buffer solution containing one or more phosphate(s), especially selected from the group consisting of H₃PO₄, H₂PO₄, HPO₄²⁻, PO₄³⁻, and salts thereof, especially sodium and potassium salts, and mixtures of the phosphates and salts thereof, especially sodium and potassium salts. The term “buffer solution” describes a mixture of substances whose pH changes significantly less when an acid or a base is added than would have been the case in an unbuffered system. Preferably, the term “buffer solution” describes an aqueous solution.

Preferably, the terms “Na₂HPO₄” and “KH₂PO₄” as used herein include the hydrates of Na₂HPO₄ and KH₂PO₄, respectively. Particularly preferably, the term “Na₂HPO₄” includes the hydrates Na₂HPO₄*12H₂O and Na₂HPO₄*2H₂O.

Preferably, the, one or more phosphate(s) in the buffer solution is/are selected from the group consisting of Na₂HPO₄, in particular Na₂HPO₄*12H₂O, Na₂HPO_{4*2}H₂O, KH₂PO₄.

Particularly preferably, the, one or more phosphate(s) in the buffer solution is/are Na₂HPO₄ and/or KH₂PO₄.

Component (B) may further include NaCl.

It is preferred that in component (A) of a gel-forming multicomponent composition according to the invention
sodium alginate (component (i)) is present in a range of from 0.5 to 10 wt.-%, preferably 0.5 to 7.5 wt.-%, more preferably 0.5 to 5 wt.-%, further preferably 0.5 to 2.5 wt.-%, more preferably 0.5 to 1.25 wt.-%; particularly preferably 0.75 to 1.0 wt.-%, based on the weight of component (ii),
and/or
Na₂HPO₄ (component (v)), if present, is present in a range of from 0.005 to 0.3 wt.-%, preferably 0.01 to 0.2 wt.-%, more preferably 0.05 to 0.1 wt.-%, based on the weight of component (ii),
and/or
KH₂PO₄ (component (vi)), if present, is present in a range of from 0.001 to 0.1 wt.-%, preferably from 0.005 to 0.05 wt.-%, more preferably from 0.0075 to 0.025 wt.-%, based on the weight of component (ii).

Preferably, 0.001 to 10 wt.-%, preferably from 0.005 to 5 wt.-%, more preferably from 0.01 to 1 wt.-%, most preferably from 0.05 to 0.5 wt.-% of phosphate buffer as described herein, in each case based on the weight of component (iii), are present in component (A) of a gel-forming multicomponent composition according to the invention.

Preferably, 0.0005 to 0.1 wt.-%, more preferably 0.00075 to 0.05 wt.-%, most preferably 0.001 to 0.01 wt.-% of one or more phosphate(s) as described herein, each based on the weight of component (iii), are present in component (A) of a gel-forming multi-component composition according to the invention.

Additionally or alternatively, it is also preferred that in component (B) of a gel-forming multicomponent composition according to the invention
NaCl (component (d)), if present, is present in a range of from 0.05 to 1.5 wt.-%, preferably 0.1 to 1.0 wt.-%, more preferably 0.25 to 0.75 based on the weight of component (b),
and/or
CaCl₂) (component (a)) is present in a range of from 0.25 to 5.0 wt.-%, preferably 0.75 to 2.5 wt.-%, particularly preferably 1.5 to 2.0 wt.-% based on the weight of component (b).

The amount of the present components influences both the speed of the cross-linking reaction and the stability and flexibility of the crosslinked gel. The preferred amounts described above ensure that a stable and flexible gel is formed as quickly as possible, which can be removed from the body in one piece if necessary.

Surprisingly, it was also found that the gel-forming multicomponent composition according to the invention has a longer shelf life. This allows storage of the individual components with little or no loss of function.

The term “sterilization” preferably describes a process by which a material or an object is freed from microorganisms, preferably from living microorganisms, and/or viruses and/or prions and/or nucleic acids, preferably plasmids, or their number is reduced and/or the microorganisms are killed. Microorganisms are preferably also to be understood as their resting stages (e.g. spores).

Additionally or alternatively, the term “sterilization” preferably includes a process after the performance of which neither living Escherichia coli nor living salmonella are present.

Preferably, the term “sterilization” describes a process after the performance of which the total number of aerobic microorganisms does not exceed a value of 103 CFU/g (CFU: colony forming unit) and/or the total number of yeasts and fungi does not exceed a value of 102 CFU/g, where the weight (in grams) refers to the goods or material to be sterilized.

Preferably, the term “sterile” describes the absence of living microorganisms, the residual content of living microorganisms in a unit of the sterilized material being at most 10-6. A residual content of 10-6 or less means that there is only one living microorganism in one million equally treated units of sterilization material.

Preferably, the term “living microorganism” describes a microorganism capable of reproducing.

Preferably, the term “sterile” additionally or alternatively describes the absence of replicable viruses or virions, wherein the residual content of replicable viruses or virions in a unit of sterilization material is at most 10-6. A residual content of at most 10-6 means that only one replicable virus or virion is contained in one million equally treated units of a sterilization material.

Preferably, the term “sterile” additionally or alternatively describes the absence of prions, preferably functional prions, wherein the residual content of prions, or functional prions, in one unit of sterilization material is at most 10-6. A residual content of at most 10-6 means that only one prion, or functional prion, is contained in one million equally treated units of a sterilization material.

Preferably, the term “functional prion” describes a prion that can change the conformation of a protein such that the protein also becomes a prion.

Preferably, the term “sterile” additionally or alternatively describes the absence of nucleic acids, preferably functional nucleic acids, wherein the residual content of nucleic acids, or functional nucleic acids, in a unit of the sterilized material is at most 10-6. A residual content of at most 10-6 means that only one nucleic acid molecule, or functional nucleic acid molecule, is contained in one million equally treated units of a sterilization material.

Preferably, the term “functional nucleic acid molecule” describes a nucleic acid molecule that can still be transcribed and/or translated.

Preferably, the term “sterile” additionally or alternatively describes an SAL of at least 1×10⁻⁶. The SAL of a sterilization process is expressed as the probability of survival of microorganisms in a product element after it has undergone the process. For example, an SAL of 10-6 indicates a probability of at most 1 non-sterile element in 1×10⁶ sterilized elements of the final product. The SAL of a process for a given product is established through appropriate validation studies (Source: Ph. Eur. 10.-10.0/5.01.01.00).

Possible sterilization processes include processes using chemical substances, using physical methods, or combinations thereof. In particular, such methods may be selected from the group consisting of steam sterilization, hot air sterilization, irradiation, gas sterilization, and sterile filtration.

In steam sterilization, the air inside the sterilization device, preferably an autoclave, is replaced by water vapor. Preferably, the material to be sterilized is brought to a temperature of at least 121° C., preferably in a range of from 121° C. to 134° C., preferably at a pressure of at least 2 bar, preferably in a range of from 2 to 3 bar. Preferably, the material to be sterilized is exposed to said conditions for at least 5 minutes, preferably at least 10 minutes, more preferably at least 15 minutes, preferably a time in the range of from 5 to 25 minutes.

Preferably, the material to be sterilized is subjected to a temperature of at least 121° C. at a pressure of at least 2 bar in water vapor for at least 20 minutes.

Preferably, the item to be sterilized is subjected to a temperature of at least 134° C. at 3 bar pressure at least in water vapor for at least 5 minutes.

Preferably, the sterilization process has an F₀ value of at least 8.

In the hot-air sterilization, the item to be sterilized is preferably subjected to a temperature of at least 160° C., preferably at least 170° C., particularly preferably at least 180° C., especially preferably at least 220° C. Preferably, the item to be sterilized is exposed to the temperature for at least 30 minutes, preferably at least 60 minutes, more preferably at least 120 minutes.

During the irradiation, the material to be sterilized is exposed to ionizing radiation. The ionizing radiation is preferably selected from UV radiation, X-ray radiation, beta radiation and gamma radiation. Preferably, the energy dose caused by ionizing radiation is at least 25 KGy.

In gas sterilization, the item to be sterilized is exposed to a gas that kills microorganisms, for example. Preferably, the gas is ethylene oxide.

In sterile filtration, the material to be sterilized is filtered. Preferably, a filter with a pore size of maximum 0.25 μm, preferably maximum 0.22 μm, particularly preferably maximum 0.2 μm, especially preferably maximum 0.1 μm is used.

Preferably, during sterile filtration, a bubble point test is carried out after filtration to check the quality of the filter.

Advantageously, sterilization of medical devices is carried out in the final packaging, as this procedure allows simpler proof of a sterile offered product and offers simpler maintenance of the (entire) manufacturing process.

Surprisingly, it was also found that the gel-forming multicomponent composition according to the invention forms a gel even in the presence of aqueous solutions, e.g., a physiological saline solution. This is typically not the case with previously known compositions or adhesives, or these cannot form a suitable gel in the presence of aqueous solutions that exhibits sufficient strength.

Further preferably, component (A) and/or component (B) of a gel-forming multi-component composition according to the invention comprises or comprise at least one dye. In this context, it is particularly preferred that the dye is physiologically harmless and thus can be used without problems in the application to/in a patient.

In the removal of undesirable particles from a patient, it has been shown that a dyed gel has several advantages. The exact amount and location of the injected component(s) can be precisely controlled. It can also be determined whether gel formation is already complete and the gel formed can be easily retrieved endoscopically. In addition, it can be determined whether the respective components are swirled due to the use of a rinsing solution or excessive dosing, which would possibly dilute the components to such an extent that proper function cannot be guaranteed.

To take advantage of such benefits, Cloutier J, Cordeiro E R, Kamphuis G M, Villa L, Letendre J, de la Rosette J J, Traxer O (2014) “The glueclot technique: a new technique description for small calyceal stone fragments removal” Urolithiasis 42 (5):441-444, describes using autologous blood. However, the formation of a blood clot from clotted blood takes significantly longer than the formation of a consolidated gel from the components as described herein. Also, the low color contrast between blood and the patient's tissue makes it difficult to use. Furthermore, the treating physician wishes to see the embedded stone fragments, as a control of the success of the procedure. However, this is not feasible in the blood clot because of the strong inherent color of the blood. However, dilution of the blood would prevent the formation of a solid blood clot or greatly reduce its strength. In addition, the autologous blood cannot be applied with the help of a fine catheter specifically in the area of the stone fragments, e.g. in a distal renal calyx (blood has too high a viscosity for this).

In addition, it is preferred that the or at least one of the at least one dye has a high contrast to the tissue of the patient in which the multicomponent composition is applied. Thus, it is preferred that the or at least one of the at least one dye does not provide a red or yellow-orange coloration.

It is further preferred that the or at least one of the at least one dye does not provide black or white coloration. This allows an even better control of success, whether the particles to be removed are already enclosed by the gel or which of the particles are already enclosed and where a gel is still needed to remove the particles.

It is also particularly advantageous if the dye does not escape from the solidified gel and/or from the or a component containing the dye. In this case, no surrounding tissue of the patient is stained and the dye can serve as an indicator for the gel and/or the component(s) containing the dye. It is therefore possible to check whether the solidified gel has been completely removed from the patient. In addition, a dyed component can be better dosed with regard to the quantity as well as the actual place of application. Also, confusion between the gel and the patient's tissue is minimized and, when the gel is subsequently removed, only the gel itself, together with the particles enclosed in it, is removed and not (endogenous) tissue of the patient that is not to be removed. Accidental injury to surrounding patient tissue is thus minimized.

It is further preferred that component (A) and component (B) comprise at least one dye and wherein the dye(s) in component (A) is/are different from the dye(s) in component (B). This has proven to be advantageous, in particular, in that it allows a color distinction to be made between components (A) and (B). It is also preferably and advantageously possible in this way to determine whether components (A) and (B) have already mixed or have been mixed, since mixing the two components preferably results in a color that is different from the color of the colored components (A) and/or (B).

The phrase “wherein the dye(s) in component (A) is different from the dye(s) in component (B)” should be understood to mean that in the case of multiple dyes in one or both of components (A) and (B), the combination of dyes of one component is not the same as the combination of dye(s). Consequently, both components may contain the same dye as long as at least one of the components also contains another, different dye.

In particular, it is preferred that one of the two components (A) and (B) comprises dextran blue. Surprisingly, it has been found that dextran blue in particular has the additional advantage that this dye does not diffuse out of the gel and therefore does not stain the surrounding tissue, which is particularly advantageous when removing the gel together with enclosed particles, but without injury to the surrounding tissue, and when checking whether the gel has been completely removed.

Particularly preferably, one of the two components (A) and (B) comprises dextran blue and the other of the two components (A) and (B) comprises riboflavin.

Preferably, in one of the two components (A) and (B) dextran blue is present in a range of from 0.01 to 1.0 wt.-%, preferably 0.05 to 0.75 wt.-%, more preferably 0.1 to 0.5 wt.-%, based on the weight of component (ii) or component (b).

Additionally or alternatively, riboflavin is present in one of the two components (A) and (B) in a range of from 0.0001 to 0.05 wt.-%, preferably 0.0005 to 0.01 wt.-%, more preferably 0.001 to 0.005 wt.-%, based on the weight of component (ii) or component (b).

The expression “based on the weight of component (ii) or of component (b)” is to be understood in such a way that the indication “wt.-%” refers to the weight of component (ii), provided that dextran blue or riboflavin is present in component (A), whereas the indication “wt.-% weight” refers to the weight of component (b), provided that dextran blue or riboflavin is present in component (B).

This is particularly advantageous as it provides a coloration of the gel that has a high contrast to the surrounding tissue of the patient. At the same time, dextran blue or riboflavin in this concentration enables particularly good success control, as described above.

Particularly preferably, component (A) and/or component (B) and/or another component (C) optionally contained in the composition has a neutral pH, preferably a pH in the range of from 6.5 to 8.0, particularly preferably a pH in the range of from 7.0 to 7.5. A neutral pH has been shown in particular to be harmless to body tissue. Moreover, the components are not or almost not degraded or damaged during sterilization, e.g. steam sterilization, autoclaving.

A composition according to the invention may additionally contain other components. For example, substances that promote gel formation and/or incorporation of the particles, may be added to components (A) and/or (B) and/or one or more further components of a composition according to the invention. Such substances may be, for example, crosslinkers to increase the stability of the gel.

According to the invention, it is therefore preferred that component (A), component (B) and/or a further component (C) optionally contained in the composition contains one or more substances for improving the crosslinking and/or the stability of the sodium alginate, in particular crosslinkers, preferably wherein the, a plurality or all of the substance(s) is/are selected from the group consisting of amino acids, (bio)polymers, sugar polymers, synthetic di- or multimers, sugar prepolymers and synthetic prepolymers.

A composition according to the invention may additionally or alternatively contain further components. For example, substances that promote gel formation and/or particle incorporation, as well as the density, may be added to components (A) and/or (B) and/or to one or more further components of a composition according to the invention.

Such substances may be, for example, substances for increasing the density of the gel components, in particular of the crosslinkable polymer, preferably of the sodium alginate. Such substances may be selected from the group consisting of (bio)polymers, sugar polymers, synthetic polymers, sugar prepolymers, sugar monomers, sugar dimers such as sucrose or glucose, synthetic prepolymers, hydrophilic copolymers, epichlorohydrin (Ficoll®) or glycerol.

According to the invention, it is therefore preferred that component (A), component (B) and/or a further component (C) optionally included in the composition contains one or more substances for increasing the density of the crosslinkable polymer from component (i) of component (A), preferably of sodium alginate, preferably wherein the, one, more or all of the substance(s) is/are selected from the group consisting of (bio)polymers, sugar polymers, synthetic polymers, sugar prepolymers, sugar monomers, sugar dimers such as sucrose or glucose, synthetic prepolymers, hydrophilic copolymers, epichlorohydrin (Ficoll®) or glycerol.

Preferably, component (A), component (B) and/or another component (C) optionally included in the composition contains one or more substance(s) for increasing the density of the crosslinkable polymer of component (i) of component (A), preferably of the sodium alginate as described herein, in an amount of from 1 to 30 wt.-%, preferably from 3 to 20 wt.-%, more preferably from 5 to 10 wt.-%, based on the total weight of component (A) or component (B) or the further component (C) optionally included in the composition. Where several of the components in the composition contain one or more such substances, the amount is preferably calculated individually for each component.

Provided that the or one of the substance(s) for increasing the density is a cross-linkable polymer according to component (i) of component (A), for calculating the weight of component (i) of component (A) or for calculating data based on the weight of the component, the crosslinkable polymer is taken into account as part of component (i) of component (A).

Preferably, the term “substance for increasing the density of the gel components, in particular the crosslinkable polymer, preferably the sodium alginate” does not refer to the crosslinkable polymer of component (i) of component (A) of the composition according to the invention that is used.

Further, the present invention relates to a composition according to the invention, for use in a method for removing undesirable particles from a patient.

The aforementioned removal of undesirable particles constitutes an invasive, surgical procedure. A purely cosmetic application is not subject to this. The removal of unwanted particles is performed in a targeted manner, the unwanted particles are deliberately removed from the body.

Undesirable particles can be particles of any kind that must or can be removed from the patient's body from a medical point of view.

Preferably, the particles are deposits, precipitates, foreign bodies and/or fragments thereof and/or splinters of endogenous structures. Deposits may be, in particular, urinary stones or kidney stones. Precipitates may be particles such as gallstones as a precipitate of bile or of the pancreas or salivary glands. Foreign bodies can be, for example, mineral fragments, metal, plastic or wood splinters that enter the body, for example, during injuries such as abrasions. The term “fragments thereof” refers to the aforementioned particle forms and clarifies that these can be crushed into smaller parts or fragments before or during removal. It is also possible here that only parts (fragments) are removed by the application, while the remaining fragments are removed in a further step or process. Splinters of the body's own structures can arise, for example, during operations such as the insertion or removal of implants and can therefore be, for example, bone splinters or milling residues. Preferably, however, this also includes splinters of other structures such as cartilage tissue or tumor fragments, for example, which have arisen during a previous or simultaneously performed surgical intervention. The term splinter includes structures of any shape and size.

Preferably, the removal of the undesired particles is performed as a process comprising the following steps:
(i) providing the sterilized component (A) and (B) and further component(s) (if present),
(ii) introducing the component (A) and (B) and further component(s) (if present) into the patient's body in an area containing particles to be removed, under conditions that, upon contact of component (A) with component (B), allow crosslinking of the sodium alginate to form a crosslinked gel that partially or completely surrounds the particles to be removed,
(iii) removing the cross-linked gel together with the particles enclosed thereby from the area of the patient's body.

Components (A), (B) and other components (if present) may be introduced simultaneously or sequentially in step (ii). Components may also be mixed prior to introduction into the body. Individual components may also be mixed prior to introduction into the body, while other components are first contacted with one or more other components in the body. Components (A), (B) and optionally other components (if present) may also be introduced into the body simultaneously, but separately, i.e. in a form separated from each other.

Preferably, the method comprises the following further step, which takes place temporally before step (ii):

Fragmenting one or more particles in the area of the patient's body so that two or more, preferably a plurality of fragments of the particle(s) are formed.

If this step is carried out, the particles may already be present as fragments, which are then in turn fragmented into fragments of their own by this step.

Preferably, the term “plurality of fragments” includes at least 5, preferably at least 10, more preferably at least 15, most preferably at least 20 fragments. If a particle that is fragmented in this step is already present as a fragment, the term “plurality of fragments of the particle(s)” refers to the particle that has already been fragmented and the fragments that arise from it.

In addition, the present invention relates to a process for preparing a composition according to the invention, comprising or consisting of the steps of:
(i) providing component (A) as described herein,
(ii) providing component (B) as described herein,
(iii) optionally, providing component (C) as described herein,
(iv) sterilizing component (A) and optionally component (B) and/or component (C) (if present) provided in the preceding. steps

What has been said above regarding the properties and advantages of the gel, the gel-forming multi-component composition, the components or the individual components of the gel-forming multi-component composition also applies accordingly to the components or the individual components in the manufacturing process. For example, as already described above, components (A) and/or (B) may contain at least one dye as described above. Particularly preferably, component (A) may include Na₂HPO₄ and/or KH₂PO₄, as described above. Alternatively or additionally, component (B) may include NaCl, as described above.

Preferably, the sterilization in step ii) of the process according to the invention is selected from the group consisting of steam sterilization, hot air sterilization, irradiation, gas sterilization and sterile filtration.

Compared to hot air sterilization, steam sterilization offers the advantage that it can also be used for biomaterials and many plastics. This is because steam sterilization does not destroy these materials (or only to a lesser extent).

Compared to irradiation, steam sterilization has the advantage that it does not (or only to a lesser extent) destroy biopolymers-compared to high-energy radiation, such as gamma or beta radiation. For the killing of microorganisms, it is indeed desirable that the biomolecules that form the basis of living organisms are destroyed. However, in the case of medical devices containing functional biomolecules in the form of biopolymers, high-energy irradiation also leads to drastic loss of function due to chain fragmentation.

Compared to gas sterilization, steam sterilization offers the advantage that medical devices can also be sterilized advantageously in their final packaging. In the case of gas sterilization with ethylene oxide, for example, this would not or only barely come into contact with the substance to be sterilized if it is to be sterilized in the final packaging.

Steam sterilization also has the advantage over sterile filtration in that medical devices can be sterilized advantageously in the final packaging. Sterile filtration is not the final step in the production and packaging of a medical device and thus is not a terminal process.

Preferably, step (iv) of the process according to the invention includes or consists of a step of steam sterilization,
preferably wherein the temperature during steam sterilization is at least 121° C., more preferably in a range of from 121° C. to 134° C.,
preferably wherein the pressure during the steam sterilization is at least 2 bar, more preferably in a range of from 2 to 3 bar,
and preferably wherein the duration of the steam sterilization is at least 5 minutes, more preferably in a range of from 5 to 25 minutes.

Particularly preferably, the sterilization in step iv) of the process according to the invention is a steam sterilization, preferably as described herein, wherein the provided component(s) is exposed to a temperature of at least 121° C. at 2 bar pressure at least in water vapor for at least 15 minutes, preferably at least 20 minutes.

Most preferably, a process wherein component (A) is provided as described herein, wherein component (A), preferably the phosphate buffer of component (A), comprises Na₂HPO₄ or KH₂PO₄, or wherein component (A), preferably the phosphate buffer of component (A), comprises Na₂HPO₄ and KH₂PO₄.

Further preferred is a method wherein component (A) is provided as described herein, wherein component (A), preferably the phosphate buffer of component (A), comprises Na₂HPO₄ or KH₂PO₄, or wherein component (A), preferably the phosphate buffer of component (A), comprises Na₂HPO₄ and KH₂PO₄, and wherein the sterilization in step ii) of the method of the invention is a steam sterilization, preferably as described herein, preferably wherein component (A) and optionally component (B) and/or component (C) (if present) is exposed to a temperature of at least 121° C. at at least 2 bar pressure in water vapor for at least 15 minutes, preferably at least 20 minutes.

Preferably, the sterilization in step iv) of the process according to the invention, in particular the processes described as preferred, has an F₀ value of at least 8, preferably at least 10, particularly preferably at least 15.

Particularly preferred is a process in which a component (A) as described herein is provided, wherein the component (A), preferably the phosphate buffer of the component (A), comprises Na₂HPO₄ or KH₂PO₄, or wherein the component (A), preferably the phosphate buffer of the component (A), comprises Na₂HPO₄ and KH₂PO₄, and wherein the sterilization in step iv) of the process according to the invention is a steam sterilization, preferably wherein component (A) and optionally component (B) and/or component (C) (if present) is exposed to a temperature of at least 121° C. at at least 2 bar pressure in water vapor for at least 15 minutes, preferably at least 20 minutes, wherein the sterilization in step ii) has an F₀ value of at least 8, preferably at least 10, more preferably at least 15.

Further, the present invention relates to a composition according to the invention, produced or producible by a process according to the invention.

In the following, the present invention is explained in more detail with reference to selected examples.

### EXAMPLES

### Example 1: Preparation of Components (A), (B) and (C)

To prepare an exemplary component (A), 5 g dextran blue and 4 g sodium alginate are dissolved in 1 L water.

The sodium alginate has a molar mass of 200,000 g/mol and a G content of 55%.

To prepare an exemplary component (B), 10 g of calcium chloride dihydrate is dissolved in 1 L of water.

To prepare an exemplary component (C), a particle suspension containing 4 to 40 mM iron (0.35 to 3.5 g per liter) is prepared in water or physiological buffer (M. Geppert et al., Nanotechnology 22 (2011) 145101). This solution is added to A or B at 1% to 50%.

### Example 2: Application of a Gel-Forming System According to the Invention for the Removal of Urinary Calculi

Access to the urinary tract lumen (e.g., the renal pelvic caliceal system) is obtained either ureterorenoscopically (through the urethra, bladder, and ureter) or percutaneously (by skin puncture on the flank). A special sheath (possibly a polymer tube with metal components) with an inner diameter of 3 to 9 mm is placed inside. An endoscope is inserted into the urinary tract lumen (e.g., the renal pelvic caliceal system) through the provided access shaft, the surgical area is inspected, and the urinary stone(s) is/are visualized. The urinary stone(s) are fragmented using, for example, a holmium laser. The large and medium-sized fragments are removed with a stone trapping instrument. A catheter is then inserted via the endoscope device (through the access) and up to 3 mL, in particular 300 to 500 μL of a component (A) according to Example 1 is applied to the area of the urinary tract (e.g. into the renal pelvic caliceal system) in such a way that A surrounds or embeds the stones or the fragments of the fragmented urinary stone(s). Thereafter, also via the catheter located in the endoscope, up to 9 mL of a component (B) according to Example 1 is applied in the vicinity of B. Active mixing of A with B is not necessary. Gel formation occurs within a few seconds to one minute. The catheter may be flushed with 0.9% NaCl solution between application of A and B. A grasping instrument is then inserted through the surgical endoscope via the access sheath. The grasping instrument is used to grasp the solidified gel in one piece or in several pieces and remove it from the body by extraction.

### Example 3: Comparison of Different Gel-Forming Compositions Before and After Sterilization

The following formulations containing sodium alginate were provided:

| | | | | | 3 (according to | |
|---|---|---|---|---|---|---|
| | 1 (comparison) | | 2 (comparison) | | the invention) | |
| Sodium alginate | 0.8 | g | 0.8 | g | 0.8 | g |
| Water | 98.3 | g | 98.9 | g | 98.0 | g |
| NaCl | 0.9 | g | — | | 0.9 | g |
| PBS | — | | 0.3 g | | 0.3 g | |
| | | | (containing 0.01 g | | (containing 0.01 g | |
| | | | NaCl) | | NaCl) | |
| pH | 7.4 | | 7.4 | | 7.4 | |
| Total | 100 | g | 100 | g | 100 | g |

The formulations were each sterilized by steam sterilization for 20 minutes at 121° C.

As described above, steam sterilization also leads to breaks in the sodium alginate chains, so that longer molecular chains convert into shorter chains. This breaking from the molecular chains can be analyzed, among other things, by measuring the viscosity. For viscosity measurement, it is described in the literature that as chain lengths shorten, the value of viscosity (usually given in units of mPa*s) also decreases. The reduction in molecular chain length is usually also accompanied by a loss of functionality.

Therefore, the viscosity of the formulations before and after steam sterilization was measured using a viscometer at 25° C.

A Brookfield-AMETEK DV2T viscometer with a CPA-41Z spindle was used for this purpose, and the temperature was kept constant using a Brookfield-AMETEK TC-550 bath thermostat. The gel-forming capacity was also measured. For this purpose, the respective components were placed together in a dish in the area of the contact surfaces of the liquids with the help of a gripper (for example tweezers) a hydrogel thread was pulled out, if possible.

The following results were obtained for the individual formulations:

| | 1 | 2 | 3 (according to |
|---|---|---|---|
| | (comparison) | (comparison) | the invention) |
| Viscosity before | 157 | 123 | 135 |
| sterilization [mPa*s] | | | |
| Viscosity after sterilization | 5 | 3 | 39 |
| [mPa*s] | | | |
| Viskosität after sterilization | 3 | 2 | 29 |
| [%] | | | |
| Gel formation before | yes | yes | yes |
| sterilization | | | |
| Gel formation after | no | no | yes |
| sterilization | | | |

The viscosity after sterilization [%] is calculated as the quotient of the viscosity after sterilization [mPa*s] and the viscosity before sterilization [mPa*s].

The viscosity of formulation 3 according to the invention was increased by a factor of 7.8 after sterilization compared with formulation 1. Compared to formulation 2, the viscosity after sterilization was even increased by a factor of 13. Gel formation after sterilization was only possible with formulation 3 according to the invention.

1-15. (canceled)

16. A sterilized gel-forming multi-component kit for forming a gel comprising:
(A) component (A) comprising:
(i) sodium alginate,
(ii) water,
(iii) NaCl in a range of from 0.3 to 1.2 wt. %, based on a total weight of component (A), and
(iv) one or more phosphate buffers; and
(B) component (B) comprising:
(a) one or more crosslinking agents for crosslinking the sodium alginate of component (A)(i), and
(b) water;
wherein a gel is formed by mixing component (A) and component (B).

17. The sterilized gel-forming kit of claim 16, wherein the phosphate buffer in component (A)(iv) includes one or more phosphates selected from H₃PO₄, H₂PO₄, HPO₄²⁻, PO₄³⁻, salts thereof, or mixtures thereof.

18. The sterilized gel-forming kit of claim 16, wherein the sodium alginate in component (A)(i) is in an amount of 0.5 to 10 wt. %, based on the total weight of component (A)(ii), and/or the one or more crosslinking agents of component (B)(a) is in an amount of 0.25 to 5.0 wt. %, based on a total weight of component (B).

19. The sterilized gel-forming kit of claim 16, wherein the cross-linking agent of component (B)(a) is CaCl₂).

20. The sterilized gel-forming kit of claim 16, wherein component (A), component (B), or both Component (A) and Component (B) include at least one dye.

21. The sterilized gel-forming kit of claim 20, wherein both component (A) and component (B) include at least one dye, wherein at least one dye in component (A) is different from at least one dye in component (B).

22. The sterilized gel-forming kit of claim 20, wherein one of component (A) and component (B) includes dextran blue and/or one of component (A) and component (B) includes riboflavin.

23. The sterilized gel-forming kit of claim 22, wherein
one of component (A) and component (B) includes dextran blue in an amount of 0.01 to 1.0 wt. %, based on the total weight of component (A)(ii) or component (B)(b), and
the other of components (A) and component (B) includes riboflavin in an amount of 0.0001 to 0.05 wt. %, based on the total weight of component (A)(ii) or component (B)(b).

24. The sterilized gel-forming kit of claim 16, wherein component (A), component (B), and/or a further component (C) optionally contained in the composition, has a neutral pH.

25. The sterilized gel-forming kit of claim 16, wherein
component (A), component (B), and/or a further component (C) optionally contained in the composition, comprises one or more substances for improving cross-linking or stability of the sodium alginate of (A)(i), and/or
component (A), component (B) and/or a further component (C) optionally contained in the composition, comprises one or more substances for increasing density of the sodium alginate of (A)(i).

26. A gel formed by combining component (A) and component (B) of the kit of claim 16.

27. A method for removing particles from a human patient comprising:
(i) providing a sterilized gel-forming multi-component kit of claim 16;
(ii) introducing component (A) and component (B) into the patient's body in an area from which the particles are to be removed such that component (A) and component (B) come into contact with one another and form a crosslinked gel that partially or completely surrounds the particles to be removed; and
(iii) removing the crosslinked gel with the particles from the patient's body.

28. The method of claim 27, further comprising fragmenting one or more particles in the patient's body prior to introducing component (A) and component (B) into the patient's body.

29. A method of preparing a sterilised gel-forming composition of claim 16 comprising:
(i) providing component (A);
(ii) providing component (B);
(iii) optionally, providing component (C); and
(iv) sterilizing the component (A).

30. The method of claim 29, wherein component (A) is sterilized with steam sterilisation.

31. A sterilised gel-forming composition produced by the method of claim 29.

32. The sterilized gel-forming multi-component kit of claim 16, wherein the sodium alginate in component (A)(i) is in an amount of 0.5 to 5 wt. %, based on the total weight of component (A)(ii), and one of the one or more crosslinking agents of component (B)(a) is CaCl₂) in an amount of 0.25 to 5 wt. %, based on a total weight of component (B).

33. The sterilized gel-forming multi-component kit of claim 16, wherein the sodium alginate in component (A)(i) is in an amount of 0.5 to 2.5 wt. %, based on the total weight of component (A)(ii), and one of the one or more crosslinking agents of component (B)(a) is CaCl₂) in an amount of 1.5 to 2 wt. %, based on a total weight of component (B).

34. The sterilized gel-forming kit of claim 22, wherein
one of component (A) and component (B) includes dextran blue in an amount of 0.05 to 0.75 wt. %, based on the total weight of component (A)(ii) or component (B)(b), and
the other of components (A) and component (B) includes riboflavin in an amount of 0.0005 to 0.01 wt. %, based on the total weight of component (A)(ii) or component (B)(b).

35. The sterilized gel-forming kit of claim 16, wherein component (A) and component (B) have a pH in the range of 6.5 to 8.
